(19) United States
(12) Patent Application Publication
(10) Pub. No.: US 2019/0216132 A1
(43) Pub. Date: Jul. 18, 2019

(54) POROUS SINTERED BODIES FOR USE AS LIQUID STORAGE AND VAPORIZER

(71) Applicant: SCHOTT AG, Mainz (DE)

(72) Inventors: Dang Cuong Phan, Aachen (DE); Matthias Rindt, Landshut (DE)

(73) Assignee: SCHOTT AG, Mainz (DE)

(21) Appl. No.: 16/247,931

(22) Filed: Jan. 15, 2019

(30) Foreign Application Priority Data
Jan. 15, 2018 (DE) 10 2018 100 749.3

(51) Int. Cl. A24F 47/00; A61L 9/03; A61M 11/04; A61M 15/06; C03C 11/00; C03B 19/06

(52) U.S. Cl. CPC A24F 47/008; A61L 9/03; A61M 11/042; A61M 2205/8206; C03C 11/00; C03B 19/06; A61L 2209/135; A61M 15/06

(57) ABSTRACT

A liquid storage is provided that includes a sintered body made of glass or glass ceramic. The sintered body has an open porosity in a range from 10 to 90%. The sintered body is a shaped body having at least two channels that are completely or partially enclosed by the glass or glass ceramic. The sintered body is provided as a vaporizer for use in an electronic cigarette and/or in medication administration devices and/or in thermally heated vaporizers for fragrances. Here, the sintered body is a liquid storage and is used with a heating element.

### CROSS REFERENCE TO RELATED APPLICATIONS

This application claims benefit under 35 USC X119 of German Application No. 10 2018 100 749.3 filed Jan. 15, 20185, the entire contents of which are incorporated herein by reference.

### BACKGROUND

### 1. Field of the Invention

The invention generally relates to a porous sintered body for storing and/or vaporizing liquids. More particularly, the invention relates to a liquid storage and a vaporizer unit or vaporizer comprising a liquid storage and a heating unit for storage and controlled release of vaporizable substances. The vaporizer unit can be used in particular in electronic cigarettes, in medication administration devices, room humidifiers, and/or in heatable vaporizers for the release of substances into room air, such as fragrances or insect repellents, for example. It can also be employed in fog or haze machines.

### 2. Description of Related Art

Electronic cigarettes, also referred to as e-cigarettes below, are increasingly being used as an alternative to tobacco cigarettes. Typically, electronic cigarettes include a mouthpiece and a vaporizer unit as well as an electrical power source operatively connected to the vaporizer unit. The vaporizer unit includes a liquid storage which is connected to a heating element.

Certain medications, especially medications for treating the respiratory tract and/or oral and/or nasal mucosa, for pain therapy and psychotherapy and/or for the treatment of epilepsy as well as immunodeficiency syndromes are beneficially administered in the vaporized form, e.g. as an aerosol. The vaporizers of the invention can be used for the storage and administration of such medication, in particular in administration devices for such medication.

Thermally heatable vaporizers are increasingly utilized to provide an ambience with fragrant substances and/or with what is known as fog or haze. This may in particular be in bars, hotel lobbies, event locations and on stages, in training facilities, for example for fire protection, and/or in vehicle interiors, such as the interiors of motor vehicles, in particular automobiles. The vaporizer unit used in this case also has a liquid storage connected to a heating element. The liquid storage contains a liquid which is usually a carrier liquid such as propylene glycol and/or glycerol, in which additives are dissolved or more generally contained, such as fragrant and flavoring substances and/or nicotine and/or medications, with the use of appropriate solvents such as water and/or alcohols. The carrier liquid is bonded to the inner surface of the liquid storage by adsorption processes. Optionally, a separate liquid storage is provided to supply liquid to the liquid storage.

Generally, the liquid stored in the liquid storage is vaporized by heating the/a heating element, desorbs from the wetted surface of the liquid storage and can be inhaled by the user and/or is released into a space. Temperatures of over 200° C. may temporarily be reached in this context.

The liquid storage therefore has to exhibit high uptake capability and a high adsorption effect, however, at the same time the liquid has to be released rapidly at high temperatures.

From the prior art, liquid storages and vaporizer units are known which include a liquid storage made of porous glasses or ceramics. The higher temperature stability of such liquid storages allows to realize a more compact design of the vaporizer and thus of the electronic cigarette as a whole.

Local vaporization can be achieved in practice by a low pressure associated with a high temperature. In an electronic cigarette, the low pressure is achieved, for example, by the suction pressure when drawing on the cigarette during consumption, so it is the consumer who regulates the pressure. The temperatures required for vaporization in the liquid storage are produced by a heating unit. Usually, temperatures of more than 200° C. are reached here in order to ensure rapid vaporization.

DE 10 2015 113 124.2 describes open-pore sintered glasses as a liquid storage for electronic cigarettes. If provided with an electrically conductive layer, they can also be used as a heating element in a vaporizer head. The vaporization space is defined by the pores of the sintered body and is therefore limited. The limited vaporization volume also limits the maximum amount of vapor.

EP 2 764 783 A1 also describes liquid storages for electronic cigarettes comprising a porous sintered body. The latter is used together with a heating coil as a vaporizer.

Heating power is mostly provided by an electric heating coil powered by a disposable or rechargeable battery. The heating power required depends on the volume to be vaporized and on the efficiency of heating. In order to avoid decomposition of the liquid due to excessive temperatures, heat transfer from the heating coil to the liquid should occur through contactless radiation. For this purpose, the heating coil is mounted as close as possible to the vaporization surface, but preferably without touching it. If, however, the coil touches the surface, the liquid is often overheated and decomposes.

This is the case when a large amount of vapor is needed in operation and the liquid transport to the surface of the vaporizer does not occur fast enough. In this case, the energy supplied by the heating element cannot be consumed for vaporization, the surface dries out and might locally heat up to temperatures well above the vaporization temperature, and/or the temperature stability of the liquid storage is exceeded. Therefore, accurate temperature adjustment and/or control is essential. However, a drawback thereof is the resulting complex structure of the electronic cigarette, which manifests itself in high production costs, inter alia. In addition, vapor generation is possibly reduced due to the temperature control, and so is the maximum possible vapor intensity.

### SUMMARY

It is therefore an object of the invention to provide a porous sintered body for use as a liquid storage, which has a shape that is optimally adapted to the particular application and offers a variety of design options. Another object is to provide a vaporizer unit for hot applications, which comprises a liquid storage and a heating unit and exhibits improved efficiency over the prior art. In particular, the invention aims to provide a high amount of vapor generated with low heating power.

The liquid storage of the invention comprises a sintered glass or glass ceramic body, and the sintered body has an open porosity in a range from 10 to 90%. The liquid storage or the vaporizer unit may also comprise a sintered body in the form of a porous ceramic.

The liquid storage stores a carrier liquid by adsorptive interactions, and the carrier liquid may contain, for example, fragrant and flavoring substances and/or medications including active substances and/or nicotine dissolved in suitable liquids. If the liquid storage is used in a vaporizer device or is part of the vaporizer device, the stored liquid is vaporized, desorbs from the wetted surface of the vaporizer, and the vapor can be inhaled by the user.

Preferably, at least 90%, more particularly at least 95% of the total pore volume are open pores. Open porosity can be determined using measuring methods according to DIN EN ISO 1183 and DIN 66133.

According to one embodiment of the invention, the sintered body has an open porosity in a range from at least 20%, preferably 20% to 90%, more preferably 50% to 80%, and most preferably in a range from 60% to 80%. Due to the high porosity, high adsorption capacity of the sintered body is ensured. According to one embodiment, for example, the sintered body is able, at a temperature of 20° C. and within an adsorption time of 3 hours, to adsorb propylene glycol in an amount of at least 50% of its open pore volume. At the same time, the sintered body has a good mechanical stability. In particular sintered bodies with a relatively low porosity exhibit high mechanical stability, which can be particularly advantageous for some applications. According to another embodiment, open porosity is from 20% to 50%.

According to one embodiment of the invention, the pores have an average pore size in a range from 1 μm to 5000 μm. Preferably, the pore size of the open pores of the sintered body is in a range from 50 to 5000 μm, preferably in a range from 50 to 1000 μm, more preferably in the range from 100 to 800 μm and most preferably in a range from 200 to 600 μm. According to one embodiment of the invention, the pores have an average pore size of at least 50 μm.

Pores of such size are advantageous, as they are small enough to generate a sufficiently large capillary force to ensure resupply of the liquid to be vaporized, especially when used as a liquid storage in a vaporizer, at the same time they are large enough to allow for rapid release of the vapor.

The sintered body is a shaped body having at least two channels. According to one embodiment, the channels extend over the entire length l of the shaped body. Thus, the channels have the same length as the shaped body or as the dimension of the shaped body in the direction in parallel or at least substantially in parallel to the channels. Thus, the channels open to at least two end faces of the shaped body, i.e. the ends of the channels are open. Open channels are particularly advantageous if vapor shall escape therefrom, i.e. if they are vaporization channels.

Alternatively, at least one of the channels may have a channel length l_{c} which is shorter than the length l of the shaped body. Depending on the arrangement of the respective channel or channels, the channel may have closed channel ends. Channels with one open end and one closed channel end are possible as well. Such channels in particular function as inflow channels for supplying the liquid to be vaporized. Liquid can thus flow into the sintered body through the open channel end. Due to the closed end of the channel, leakage of the liquid is prevented at the same time, and/or transfer of the liquid into the evaporation channels is prevented or at least minimized.

The channels increase the surface area of the sintered body, so that the liquid storage has a large contact area for uptaking the liquid. As a result, quick uptake of the liquid is possible. When the liquid storage is used in a vaporizer, the channels at the same time increase the surface area for exit of the vapor.

The channels are defined by the surrounding material of the sintered body. The channel can be completely enclosed by the material of the sintered body along its longitudinal axis. According to one embodiment, the channels are bores or slits within the sintered body.

Alternatively or additionally, the sintered body may also have channels which are only partially enclosed by the material of the sintered body. According to this embodiment, the channels may be formed in particular as grooves in one or more lateral or outer surfaces of the sintered body.

One embodiment of the invention contemplates that at least one channel is formed as a bore, preferably a circular or ellipsoidal bore, or as a slit. Depending on the application and design specifications for a vaporizer design, other geometrical shapes of holes, grooves and slits are conceivable, for example with a polygonal cross section, although these may possibly be more complex to produce.

The sintered body may have different shapes, depending on its use. The particular shape can already be determined by the shape of the green body prior to sintering. Due to the mechanical stability of the sintered body, mechanical processing following the sintering process is also possible, for example by grinding, cutting, or drilling operations.

The sintered body may be formed in one piece. Another embodiment contemplates that the sintered body is made up of at least two individual parts which can be connected to one another. Alternatively, it is also possible for the individual parts of the sintered body to be installed separately from each other, for example in a vaporizer, i.e. without being frictionally, positively, or cohesively connected to each other.

According to one embodiment of the invention, the sintered body is in the form of a cylinder having a length l. In this embodiment, the channels extend in parallel or substantially in parallel. One of the channels is defined by the inner circumferential surface of the cylinder. This channel will also be referred to as a first channel below. The liquid storage has at least one further, second channel in this case.

One embodiment contemplates that the liquid storage has at least two second channels, preferably at least three second channels, and more preferably at least four second channels. Preferably, the second channels are arranged symmetrically around a first channel.

According to one embodiment, the second channels have a closed circumferential surface which is defined by the material of the sintered body.

A further embodiment contemplates that the second channels do not have a closed circumferential surface defined by the material of the sintered body. Such second channels are thus located in the outer lateral surface of the hollow cylinder and have an opening along their longitudinal axis.

The sintered body of this embodiment preferably has a star-type or star-like shape. In this case, the number and shape of wings or points of the star is determined by the number and cross-sectional shape of the second channels. According to one embodiment, the sintered body has 2 to 20, preferably 4 to 10 star points or wings.

The second channels may have a circular or ellipsoidal cross section in this case. Alternatively, the second channels may have a triangular or substantially triangular cross section. Alternatively, the second channels may also have other polygonal shapes. Helical channels or channels in the form of annular cuts in the peripheral area of the hollow cylinder are also possible. Corners or edges of the wings or points of the star may be rounded or be not sharp, for constructive-, material- and/or manufacturing-related reasons.

The circumferential surface of the channel comprises two side surfaces of the sintered body in this case, which are defined by the wings or points of the star shape. In this case, the angle x between the center lines of the respective wings or points of the star is from 10 to 180°, preferably from 15 to 90°, more preferably from 30 to 60° and most preferably from 30 to 45°. According to one embodiment of the invention, the sectional area of the star points defined by the second channels decreases from inside outward.

In a preferred embodiment, the star-shaped sintered body has at least five, more preferably at least six, and most preferably at least eight second channels. The angle x is preferably 40° to 75° in this case.

The angle x is identical for all second channels of the sintered body. Thus, the second channels are arranged symmetrically. However, embodiments in which the second channels have different angles x and/or in which the perimeter is not completely covered by points or wings of equal or different angular spacing are also possible.

According to another embodiment, the sintered body has a cuboidal shape. In this case, the channels may be aligned parallel or perpendicular to the cuboid's edge having the greatest edge length.

For example, one embodiment contemplates a cuboid sintered body having channels that are aligned parallel or substantially parallel to the cuboid's edge with the greatest edge length. Such an arrangement allows to realize liquid storages with extra-long channels.

If, on the other hand, the channels are oriented perpendicular or substantially perpendicular to the cuboid's edge with the greatest edge length, then many channels with a comparatively short channel length can be obtained.

In this case, the channels may be formed as channels with closed lateral surface, i.e. the channels are located inside the cuboid sintered body. Vaporizers comprising a liquid storage of this embodiment can exhibit high vapor outputs, due to the long channels and the associated large vaporization surface area.

Alternatively or additionally, the sintered body may have open channels. In this case, a first channel may be arranged at an angle relative to a second channel, for example. In particular, the first and second channels may be arranged orthogonally to one another. The one or more second channels may be used to ventilate the sintered body. In this way, individual regions of the sintered body may be provided with air channels.

Another subject matter of the invention is a vaporizer unit for hot applications, which comprises a sintered body according to the invention as a liquid storage. The vaporizer is particularly suitable for use in an electronic cigarette, in medication administration devices, or in thermally heated fragrance vaporizers. The use for so-called fog machines which generate rather large amounts of vapor is possible as well. The vaporizer unit comprises a heating element. The heating element is preferably arranged directly on the surface of the sintered body.

The arrangement of the heating element directly on the sintered body is advantageous because the heating element mounted directly on the liquid storage requires less energy for vaporization. This saves battery power of the electronic cigarette. In addition, better temperature control can be achieved. Moreover, direct contact is also advantageous in terms of design options, for example in an electronic cigarette.

Furthermore, the sintered body may be shaped such that the vaporizer can be adapted to the geometric requirements of an electronic cigarette. Also, various design options are possible for the electronic cigarette in this way, which are no longer limited by the geometry of the vaporizer. For example, flat vaporizer are possible, for example in the form of a polygon or a disc.

Moreover, the electronic cigarette may have a more compact design, or the additional space available within the electronic cigarette may be used for other functions. Furthermore, the heating power can be influenced through the geometry and the dimensions of the heating element.

One embodiment contemplates for the heating element to be installed and/or applied in the form of a metal foil, a metal wire or, preferably, an electrically conductive coating. Due to the high temperature stability of the sintered body, it can be positioned very close to the heating element.

When an electrical voltage is applied, high temperatures are generated in the vaporizer by the electrically conductive coating, so that the carrier liquid is vaporized, desorbs from the wetted surface of the vaporizer, and the vapor can be inhaled by the user or is released into a space.

According to one embodiment of the invention, the heating element is in the form of an electrically conductive coating which is bonded to the surface of the sintered body, preferably by a material bond. Not only the pores on the lateral surfaces of the porous sintered body can be provided with the electrically conductive coating, but also the pores in the interior of the sintered body. Thus, the open pores throughout the volume of the sintered body are provided with the electrically conductive coating. Consequently, when a voltage is applied to the sintered body coated according to the invention, a current will flow through the entire volume of the sintered body which will therefore be heated throughout its volume. Hence, the electrically conductive coating is deposited on the surface area of the sintered body and bonded to the surface of the sintered body. The electrically conductive coating is thereby lining the pores which are located in the interior of the sintered body, so that when at least a portion or section of the sintered body is electrically contacted and powered, a current will flow at least partially through the interior of the sintered body and thereby heat the interior of the sintered body.

Thus, in this embodiment of the invention, heating is achieved throughout the entire current-carrying volume of the sintered body and accordingly the liquid to be vaporized is vaporized throughout the volume of the sintered body. Thus, the vapor is not only produced locally on the surfaces defining the lateral surfaces of the sintered body, but also inside the sintered body. The electrically conductive coating is applied on the surface area of the sintered body, at least partially and/or in sections thereof, and therefore forms at least part of the pore surface thereof.

In contrast to vaporizers which have a localized heating device such as a heating coil or an electrically conductive coating only on the lateral surfaces of the sintered body, capillary transport to the surface of the sintered body is not necessary. This prevents the vaporizer from running dry due to not enough capillary action and thus from local overheating. This has an advantageous effect on the service life of the vaporizer unit. Furthermore, in the event of local overheating of the vaporizer, decomposition processes of the liquid to be vaporized might be caused. This might be problematic, on the one hand because the content of active substance of a medication to be vaporized is reduced, for example. On the other hand, decomposition products are inhaled by the user, which may imply health risks. In the present vaporizer, by contrast, this risk does not exist.

The electrically conductive coating may in particular comprise or consist of a metal such as, for example, silver, gold, platinum, or chromium, or a metal oxide. In one embodiment of the invention, the metal oxide is a metal oxide selected from the group consisting of indium tin oxide (ITO), aluminum-doped zinc oxide (AZO), fluorine tin oxide (FTO), and antimony tin oxide (ATO). Metal oxides have proved to be particularly advantageous here, because of their good adhesion to glass and due to the good wetting behavior of the liquid to be vaporized on the metal oxide. Moreover, the aforementioned metal oxides, in particular ITO, exhibit high chemical and mechanical stability and are insoluble in water and alcohol, so that they are inert towards the solvent of the liquid to be vaporized. In addition, the metal oxides mentioned above are stable under temperatures of up to 2000° C. Preferably, the coating contains ITO and/or is an ITO coating.

According to one embodiment, the sintered body is in the form of a hollow cylinder of length l and has at least two channels. The channels extend parallel to the length l of the hollow cylinder or of the coated sintered body.

One embodiment contemplates that the coated sintered body is connected to a current and/or voltage source such that the current flows through the cylinder from one end face to the other end face. Hence, electrical connection is established on the end faces of the hollow cylinder and can be achieved by connecting two metallic contact plates, mechanically (frictionally) or by a solder connection or soldering of terminals (by a material bond), for example. In some cases, the establishing of electrical connection can be promoted or prepared by using electrically conductive pastes. For better bonds to the electrical terminals, it is also possible to apply a second electrically conductive layer to the areas of the sintered body to be electrically contacted, for example a conductive and/or solder layer or paste.

Since the two end faces lie parallel to each other, the current flows uniformly through the cylinder, so that uniform heating power is generated within the vaporizer.

The channels serve as a vaporization space in which the liquid exits from the circumferential surface of the channels and is vaporized. The amount of vapor that can be produced depends on the size of the circumferential surface and increases with increasing surface area.

Suction pressure and flow rate can be adjusted through the diameter of the channels and through the channel length. A high suction pressure can be achieved by small diameters and/or long channels. The volume flow increases accordingly with increasing diameter and decreasing channel length.

In the case of a hollow cylinder with only one centrally located channel, the wall thickness of the hollow cylinder determines the transport path of the liquid to be vaporized to the exit surface and thus also the efficiency of the vaporizer. With increasing wall thickness of the vaporizer, the transport path to the exit surface increases and the efficiency of the vaporizer decreases. This can be problematic especially in large vaporizers which have a correspondingly large wall thickness.

If large amounts of vapor and/or a high volume flow are required, it is usually possible in the case of a hollow cylinder that has one channel to increase the volume of the vaporizer and the vapor exit surface. However, increasing the circumferential surface of the channel, i.e. the inner surface of the hollow cylinder, results in a decrease of suction pressure. This may entail an unfavorable mix of little vapor with a lot of air in the interior of the hollow cylinder, which may have an adverse effect on a uniform release of the liquid to be vaporized.

An increase of the length of the vaporizer will also result in an increase of the amount of vapor, however, electrical resistance will increase as well due to such increase in length, so that electric power is reduced.

Therefore, an inventive vaporizer has at least two channels. According to one embodiment, the coated hollow cylinder has at least one first and at least one second channel, which have closed circumferential surfaces. Thus, the channels are located inside the sintered body and have openings at the end faces of the hollow cylinder. Preferably, the inner circumferential surface of the hollow cylinder is defined by the first channel. So, the first channel preferably defines the center of the hollow cylinder.

The second channels are preferably arranged symmetrically or at least substantially symmetrically with respect to the first channel. With the second channels, the vaporization surface area is increased compared to a respective cylinder that has only one channel, without need to increase the total diameter of the hollow cylinder. Accordingly, the transport path of the liquid remains short, so that the additional channels do not adversely affect the efficiency of the vaporizer. Rather, the additional channels reduce the transport path of the liquid from the outer lateral surface to the circumferential surface of the channel. This increases the amount of vapor that is released from the vaporizer and reduces energy consumption.

Furthermore, since the length of the cylinder does not have to be increased, the specific electrical power of the vaporizer remains the same. Suction pressure can be adjusted through the number of channels and the diameters of the channels.

Another embodiment contemplates a different mode of electrical contacting of the vaporizer. In this case, one electrical terminal or electrical contact, for example the positive terminal, is placed in the first channel, while the outer lateral surface of the cylinder then defines the other electrical terminal of the heating element, for example the negative terminal. In case of electrical contacting with the positive terminal inside the first channel, the current will flow from the center of the cylinder towards the outer surface thereof. Furthermore, the second channels function as exit areas for the vapor. According to one embodiment, the sintered body may additionally have a second electrically conductive layer, at least on portions of the surface, for improving electrical contacting.

If the negative terminal is disposed on the outer lateral surface, it is advantageous if the coated sintered body contacts the housing of the vaporizer since the housing is usually connected to the negative terminal of the power source or voltage source. Therefore, electrical insulation will not be necessary, in contrast to the case of electrical contacting at the end faces.

For electrical insulation, in particular in electronic cigarettes, an intermediate layer of electrically insulating material is used between the housing and the heating element, i.e. the coated vaporizer, for example nonwoven or fibrous materials such as cotton, glass wool, cellulose, or wool. However, such insulation is not stable in shape, so that the heating element might unintentionally contact the housing. This is especially the case when very large heating elements are used. If the cylinder is electrically contacted at the end faces, such heating element-to-housing contact will result in a short circuit.

The electrical contacting as described above with current flow of from the interior of the cylinder towards the outer lateral surface is therefore particularly suitable for vaporizers with large heating elements.

Another advantage of this electrical contacting is that the current flow within the heating element is independent of the length of the cylinder. Hence, the vaporization volume can be increased by increasing the length of the cylinder, without thereby increasing electrical resistivity. Thus, even with an increase in the length of the hollow body, the specific heating power will remain constant in the volume. This allows to provide very long vaporizers with small diameters and high vapor output.

In case of an electrical contacting as described above, the specific heating power is inversely proportional to the diameter of the vaporizer. Vaporizers with small diameters as used in electric cigarettes, for example, have a very high heating power in case of this electrical contacting. The heating power, in turn, may also be defined by the thickness of the electrically conductive coating and is proportional to the layer thickness. Hence, small vaporizers which shall have a heating power in a range from 8 to 80 W which is typical for electronic cigarettes, only need a comparatively thin electrically conductive coating. In particular when using expensive coating materials such as ITO, this is an economic advantage.

Another advantage of electrical contacting with one of the electrical terminals or electrical connections or contacts and in particular the positive terminal disposed in the first channel is that the current flow can be selectively directed within the coated sintered body. So, in contrast to electrical contacting end face to end face of the cylinder, it is furthermore possible to have inhomogeneous distributions of the current flow.

In this case, the current flow can be directed in particular in dependence of the position of the second channels and their cross-sectional shape. The current flow can also be spatially directed in dependence of the placement of the second electrical terminals, for example the negative terminals.

In case of a cylinder that has only one first channel and no further channels, and in which the negative terminal is formed by the entire outer lateral surface of the cylinder, the electrical currents from inside outward will be the same in all directions. Generally, the current and so the heating power as well decreases from inside outward in the vaporizer. However, an inverse power distribution, i.e. an increase in heating power from inside outward is more advantageous for vaporization. This is due to the fact that more liquid is supplied in the outer regions and so more power is required for vaporization in the outer regions.

This can be achieved, for example, by placing the negative terminal only in regions of the outer lateral surface of the cylinder which are located at a small distance from a second channel. As a result thereof, the current flow and thus also the heating power will be maximal in the regions of the sintered body around the second channels.

Concentrating the heating power in the regions around the second channels is advantageous because more heating power is required there due to the high vaporization. The remaining areas of the coated sintered body will have a lower temperature in this case.

In addition to the location of the negative terminal, the cross-sectional shape of the second channels also has an influence on current distribution in the coated sintered body. For example, channels with an elongated or ellipsoidal cross section aligned in the direction of the negative terminal will also result in a concentration of current flow in the regions of the sintered body adjacent to the second channels.

One embodiment contemplates that the second channels are only partially enclosed by the material of the sintered body. Thus, the second channels do not have a closed circumferential surface but are open on at least one side. Preferably, in this case, the second channels have a cross-sectional shape tapering towards the center of the cylinder. So, in this embodiment the second channels widen towards the outer surfaces of the sintered body.

In terms of current flow distribution in the sintered body it has been found particularly advantageous to have second channels of V-shaped or substantially V-shaped cross section. V-shaped cross section may also be understood as meaning a triangular or substantially triangular cross section in this case. The two sides of the open channel are defined by the material of the sintered body. The angle x enclosed by the center lines of the respective two star points or wings is preferably less than 45°.

According to one embodiment of the invention, the angle x is in a range between 30° and 60°.

Depending on their number, their location, and their cross section, the sintered body may thus have a star-shaped or partially star-shaped cross-sectional shape. A respective wing or point of the star shape is defined by two second channels. Sintered bodies with at least 4, preferably at least 6, and most preferably at least 8 second channels have proved to be particularly advantageous here.

In one embodiment of the invention, the cross section of the second channels is selected such that the sectional area of the star point decreases with increasing distance from the first channel.

In case of star-shaped heating elements it has been found to be particularly advantageous in terms of the spatial distribution of the heating power within the heating element to place one electrical terminal, for example the positive terminal, in the central first channel. The other electrical terminal is alternately positioned over every second of the second channels in this embodiment, so that the respective second channels are spatially closed by the other terminal. The channel thus has a closed circumferential surface. Preferably, the positive terminal is placed in the first channel. In this exemplary embodiment, part of the lateral surface of the respective channel is defined by the material of the coated sintered body, and part thereof by the material of the negative terminal. The so formed closed second channels are therefore spatially separated from the liquid in the liquid container and serve as vaporization chambers. The open second channels provide a large contact surface of the sintered body to the liquid. Thus, this embodiment provides for a rapid uptake of the liquid by the coated sintered body and for a rapid release through the large vapor exit surface, so that such vaporizers have a high efficiency. Furthermore, due to the specific placement of the electrical terminals, heating power is concentrated in the points or wings of the star shape. This is advantageous since in this way the heating power is greatest in the vicinity of the vaporization chambers.

One embodiment of the invention contemplates that the vaporizer has at least one third channel. The third channel extends transversely across the sintered body, preferably perpendicular relative to at least a first channel, and preferably it has a smaller diameter or a smaller cross-sectional area than the first channel and the second channel. The third channel in particular serves as an inlet opening for the fluid into the liquid storage. The third channel may in particular have a slit-like or circular shape. The third channel improves the uptake and transfer of the liquid into the vaporizer. In this case, the third channel is preferably not in communication with the first and second channels and is closed towards the vaporization space.

According to one embodiment of the invention, the vaporizer may have a plurality of heating zones which are defined by different terminals. These terminals may be separately connected electrically, so that the heating zones can be controlled individually. For example, the individual heating zones can be operated at different heating powers. If the heating zones are disposed at different positions of the outer lateral surface of a vaporizer in the form of a cylinder, for example, it is possible to achieve a temperature gradient in the vaporizer in this way. It is also possible to selectively connect or disconnect individual heating zones for controlling purposes, i.e. for increasing or reducing the heating power and hence the amount of vapor or dosage of active substances.

According to another embodiment of the invention, the coated sintered body has a cuboid or at least substantially cuboid or a polygonal cross section. The cuboid has edges a, b, and c, and the channels extend parallel to the edge a. The channels have a closed circumferential surface which is defined by the material of the coated sintered body. So, these are closed channels. The channels may be aligned parallel or perpendicular to the edge of the cuboid, which has the largest edge length.

For example, one embodiment contemplates a cuboid coated sintered body including channels that are aligned parallel or substantially parallel to the edge of the cuboid with the largest edge length. With this arrangement, liquid storages with extra-long channels are feasible.

If, on the other hand, the channels are aligned perpendicular or substantially perpendicular to the edge of the cuboid having the largest edge length, then many channels with a comparatively short channel length can be obtained.

In this case, the channels may be in the form of channels having a closed circumferential surface, i.e. the channels are located inside the cuboid vaporizer. Vaporizers with a liquid storage of this embodiment may have a high vapor output performance due to the long channels and therefore large vaporization surface area.

Alternatively or additionally, the coated sintered body may have open channels. In this case, a first channel may be arranged at an angle relative to a second channel, for example. In particular, the first and second channels may be arranged orthogonal to each other. In this way, individual regions of the sintered body may be provided with air channels.

Electrical contacting may be established via two opposite faces of the cuboid, which extend parallel to the channels. So, the faces serve as a positive or negative terminal.

Alternatively, two opposite faces of the cuboid serve as a negative terminal. In this embodiment, the positive terminal is located inside the sintered body. The channels serve as vaporization chambers and are preferably arranged equidistant from the first channel.

Another embodiment contemplates a coated sintered body including channels that have a closed circumferential surface, which circumferential surface is defined by the material of the coated sintered body. The terminal contacts are disposed such that when a voltage is applied the current flow in the sintered body will be locally limited to those regions of the sintered body which include the channels. This is advantageous, since in this manner the heating power is concentrated in the regions in which vaporization takes place, while the remaining regions of the sintered body are at a lower temperature. With an arrangement of the terminals on or in the sintered body it is thus possible to create zones within the sintered body, which exhibit a high heating power and so are at a higher temperature, so that it is there where vaporization preferably takes place. This includes preferably the channels of the sintered body, which present a large surface area for exit of the vapor. Other regions of the sintered body are not heated, or only weakly or less. They serve as storage regions. In this way, the liquid storage and the vaporizer can be implemented in a single materially bonded sintered body. In addition to a compact design, such vaporizer combinations are moreover leak-proof.

The channels may be arranged around a vaporization region. In a preferred embodiment, the sintered body has at least four channels. The channels are arranged in the sintered body in at least two rows, and the terminal contacts are placed such that the current flows through the regions of the sintered body around the channels and such that there is no or only a weak current flowing in the region between the rows of channels, and evaporation takes place only in the current-carrying regions of the sintered body. Alternatively, the channels may be arranged in a ring around the vaporization region. One embodiment of the invention contemplates a sintered body which has only the regions around the channels provided with an electrically conductive coating. Thus, only the coated region is electrically conductive and heatable, while the remaining regions of the sintered body serve as a storage region.

### DESCRIPTION OF THE DRAWINGS

The invention will now be explained in more detail by way of exemplary embodiments and with reference to the figures, wherein:

FIG. 1 is a schematic view of a vaporizer unit of an electronic cigarette;

FIG. 2 is a schematic view of an exemplary embodiment of a sintered body for use as a liquid storage;

FIG. 3 is a schematic view of an exemplary embodiment of a vaporizer including a cylindrical sintered body;

FIGS. 4 and 5 show further exemplary embodiments including star-like sintered bodies;

FIG. 6 is a schematic view of a cuboid vaporizer in an electronic cigarette;

FIG. 7 shows a further exemplary embodiment of a sintered body;

FIG. 8 illustrates the use of the sintered body shown in FIG. 7 as part of a vaporization device in an electronic cigarette;

FIGS. 9 to 12 show cuboid vaporizers with different electronic contacting modes;

FIGS. 13 and 14 show refinements of the invention comprising a plurality of heating zones;

FIG. 15 illustrates the opening angle between two points of a star-shaped liquid storage; and

FIG. 16 shows a variant of the embodiment illustrated in FIG. 14.

### DETAILED DESCRIPTION

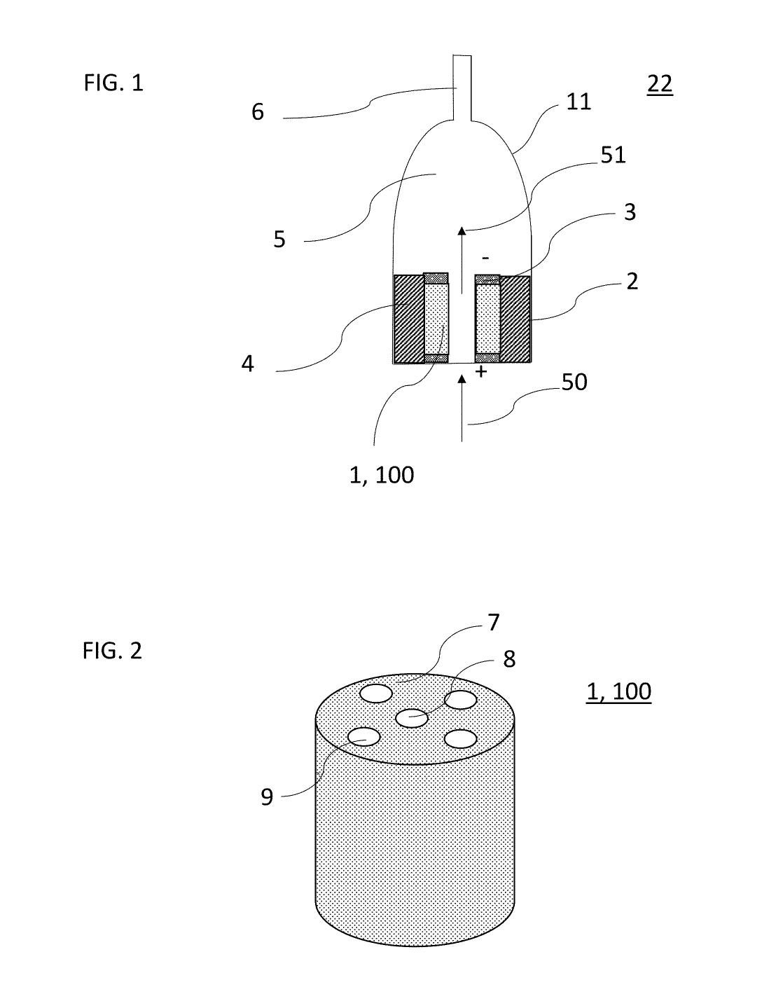

FIG. 1 schematically shows the configuration of a vaporizer head 22 of an electronic cigarette comprising a vaporizer 1. Here, the vaporizer 1 is in the form of a cylinder that includes a channel 50 and is in contact with the liquid 2 to be vaporized. The channel 50 is in the form of a bore, that is to say an enclosed channel. Electrical contacting to an electrical power source is achieved through abutting contact using two metallic contact plates 3, or by soldering to the wire line. In addition to solder, a thin silver layer, e.g. made of silver paste, on the end face is useful for soldering, for a more stable contact between wire and vaporizer. Since these surfaces 3 lie parallel to each other, current flows uniformly through the cylinder and generates uniform heating power throughout the vaporizer. When the vaporizer 1 is installed in vaporization chamber 5, its diameter should be equal to the length of standard spiral or wick (5-7 mm), so that the vaporizer has good contact to the insulating wool 4. The liquid is sucked into the vaporizer through the contact interface between wool and vaporizer (outer lateral surface of the cylinder) and is transported by suction pressure during inhalation and by capillary force. A wool layer 4 is used as an electrical insulation of the vaporizer 1 to the housing 11. The channel of the vaporizer functions as a vaporization zone. The generated vapor 51 can be inhaled by the user via mouthpiece 6. Each time the vaporizer is switched on, it is heated and the amount of liquid stored in its volume is vaporized, and liquid is resupplied. Vapor dosage can be adjusted through the size and porosity of the vaporizer. Accordingly, as it stores the liquid, the vaporizer 1 defines a liquid storage 100 according to the invention.

FIG. 2 is a schematic view of an embodiment of the sintered body 7 according to the invention, which is designed as a hollow cylinder that includes additional channels. Here, the first channel 8 defines the inner circumferential surface of the hollow cylinder. The second channels 9 are arranged around the first channel 8, preferably symmetrically or uniformly distributed. The channels are closed channels, i.e. they have a closed circumferential surface which is defined by the material of the sintered body. When provided with an electrically conductive coating (not shown), the sintered body can be used as a heating element in a vaporizer. In this case, the channels increase the size of the exit surface and at the same time reduce the transport path for liquid from the outer lateral surface to the channel 9. Suction pressure can be preset as desired through the number and diameter of the bores. Due to the significantly shorter transport path, the amount of released vapor increases and so does effectiveness. Energy consumption is reduced.

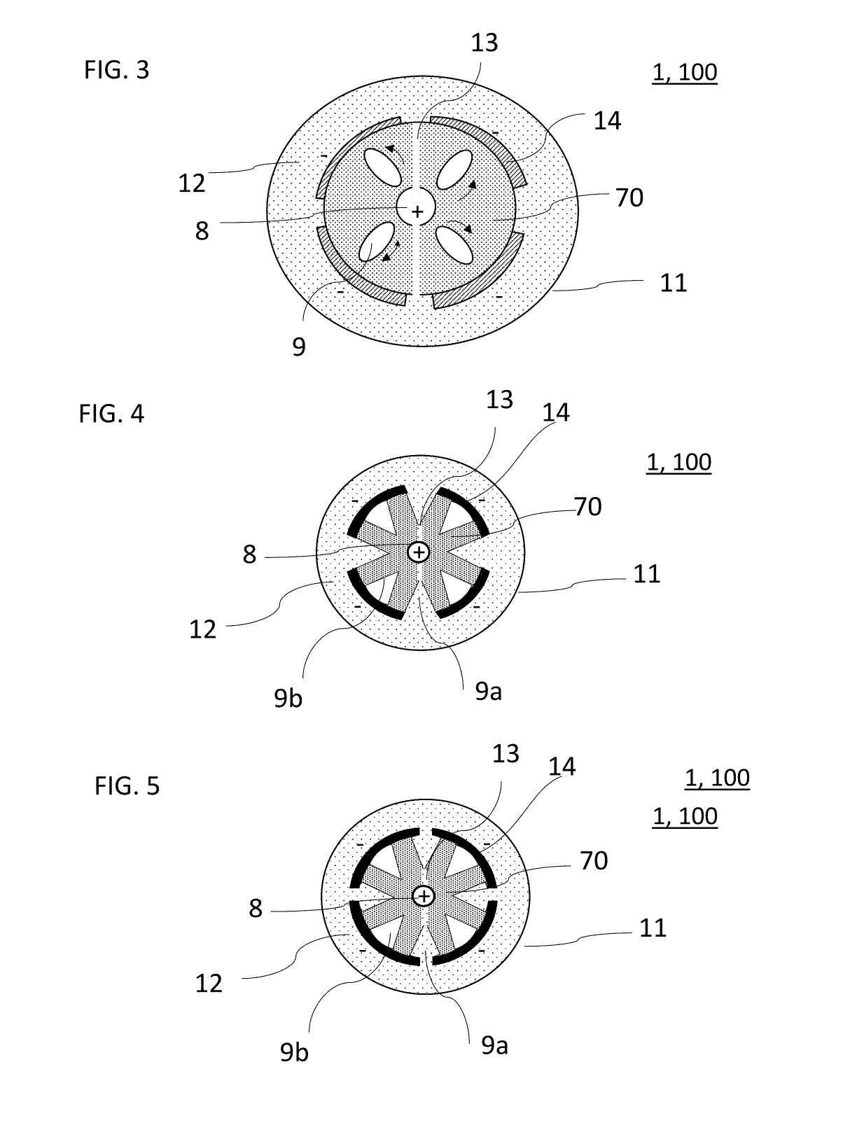

FIG. 3 illustrates a vaporizer 1 comprising a sintered body 70 with an electrically conductive coating, with electrical contacting implemented from the inside to the outside. For this purpose, the first channel 8 is used as a positive terminal. The second channels 9 are arranged symmetrically around the first channel 8 and have an ellipsoidal cross section. The negative terminal is spatially limited to the areas around the second channels 9. Current flow is symbolized by arrows 10. Due to the second channels 9 and depending on the position of the negative terminal, current flow is no longer uniform. In case the negative terminal is arranged as shown, the current flow 10 will have maximum strength around the circumferential surface of the second channels 9 and so heating power will be maximal there. With such a geometry and arrangement of the electrical terminals it is possible to selectively influence power distribution and therefore vaporization. The heating power should generally be slightly higher around the second channels 9 than in the other regions of the vaporizer, since more energy is required there due to the high vaporization. The other regions are colder in this design. In addition, the coated sintered body 70 has a third channel 13. It has a slit-like shape and functions as a liquid supply to inner regions of the sintered body. In this way it is possible to increase the uptake of liquid 12 to be vaporized in the liquid storage 100 defined by the vaporizer 1.

FIGS. 4 and 5 show exemplary embodiments with star-shaped vaporizers 1. Here, the sintered body has a first channel 8 which defines the inner circumferential surface of the hollow cylinder and accommodates the positive terminal. The second channels 9a, 9b have a V-shaped contour and are open channels with respect to the sintered body, i.e. they do not have a continuous circumferential surface defined by the material of the sintered body, but rather have an open longitudinal side or are open laterally. The cross-sectional shape of the second channels gives the coated sintered body 70 a star-like shape comprising star wings 70a. The sectional area of the star wings 70a decreases outward. Every second channel is connected to the negative terminal 14, in an alternating fashion, so that the negative terminal 14 forms part of the circumferential surface of the respective second channel, so that this channel has a closed circumferential surface which is defined partially by the material of the sintered body 70 and partially by the negative terminal 14. Thus, the negative terminal 14 spatially shields the respective channel 9b from the liquid to be vaporized.

Thus, channel 9b constitutes a vaporization zone. With that arrangement of the electrical terminals in combination with the cross-sectional shape of the second channels 9a it is possible to concentrate the heating power in the wings 70a of the vaporizer.

Channels 9a have an open V-shaped contour and thus provide for a large contact surface area to the liquid 12. Thus, the vaporizers shown in FIGS. 4 and 5 have both, large contact surfaces areas to the liquid, and also large exit surface areas for vapor. This provides for rapid uptake and rapid release, and hence for a very high efficiency of the vaporizer.

In the vaporizer shown in FIG. 5, the size of the opening to the liquid container is reduced by the size and position of the negative terminal such that there is minimal liquid pressure in the vaporization chamber and so the risk for leakage of the liquid is minimized.

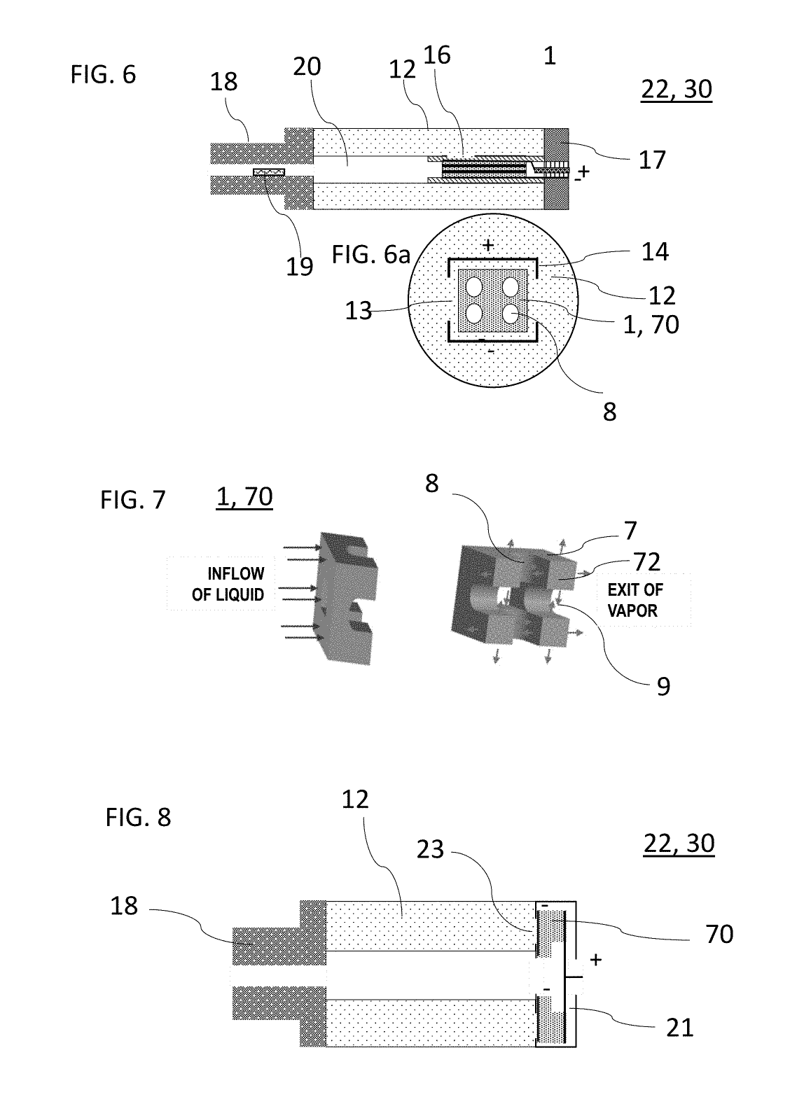

FIG. 6 shows a vaporizer head 22 of an electronic cigarette 30 comprising a vaporizer 1 in the form of a cuboid 70. Typical dimensions of cuboid or block-type vaporizers in electronic cigarettes are dimensions of B×H×L=5×5×(3 to 5) mm³. The vaporizer shown in FIG. 6 has a greater length L than width B.

The cuboid vaporizer 1 of the invention can be adapted to the design in terms of shape and size such that it fits exactly in the position of standard wick and thus can replace the previously used wicks with heating coil by simple replacement of the components. In e-cigarettes and in vaporization chambers, the vaporizer 1 is installed such that the lateral surfaces of the vaporizer contact the wool layer (with liquid) 13 and uptake liquid 12 from the wool thereby. Liquid 12 is transported to the center, where it is heated and vaporized when the e-cigarette is switched on.

The contacting with contacts, or +/−terminals, is shown in FIG. 6a. Metal plates or soldered metal wires can be used as terminals 14. For improving the connection it is possible to use solder tin, a thin layer of silver paste, or other metal coatings, for example in the form of a second electrically conductive coating. The exit surfaces for vapor are then circumferential surfaces of the channels 8.

FIG. 7 shows a coated sintered body 1 in the form of a cuboid 70 that has first and second channels 8, 9. Channels 8, 9 are open channels and have a U-shaped cross-sectional shape. Here, the first channels 8 are orthogonal to the second channels 9. This gives the coated sintered body 7 feet 72 with air channels. When powered, the electric current will concentrate in the feet and generate maximum heating power there. By virtue of channels 8, 9, a large exit surface area is given in this region for the vapor. In addition, the air channels serve as a thermal insulation and retain heat in the vaporizer.

The region of the sintered body 7, which is close to the liquid container when used in a vaporizer, has no channels. Therefore, this portion of the sintered body is more solid. This is advantageous, since in this way the sintered body can fulfill a sealing function and can prevent leakage of the liquid. In addition, this region exhibits good thermal insulation, due to the solid construction. Due to its larger cross section, the electric current is distributed and the heating power is accordingly reduced in this region.

FIG. 8 shows the use of the vaporizer illustrated in FIG. 7 in an electronic cigarette 30. Here, the vaporizer 70 is located directly below the liquid container 12. It serves as a lid of the liquid container 12. Liquid inflow to the vaporizer occurs through small openings in the bottom of the container 23. Due to the sealing function of the vaporizer, an inflow into the vaporizer is promoted, but leakage of the liquid is prevented.

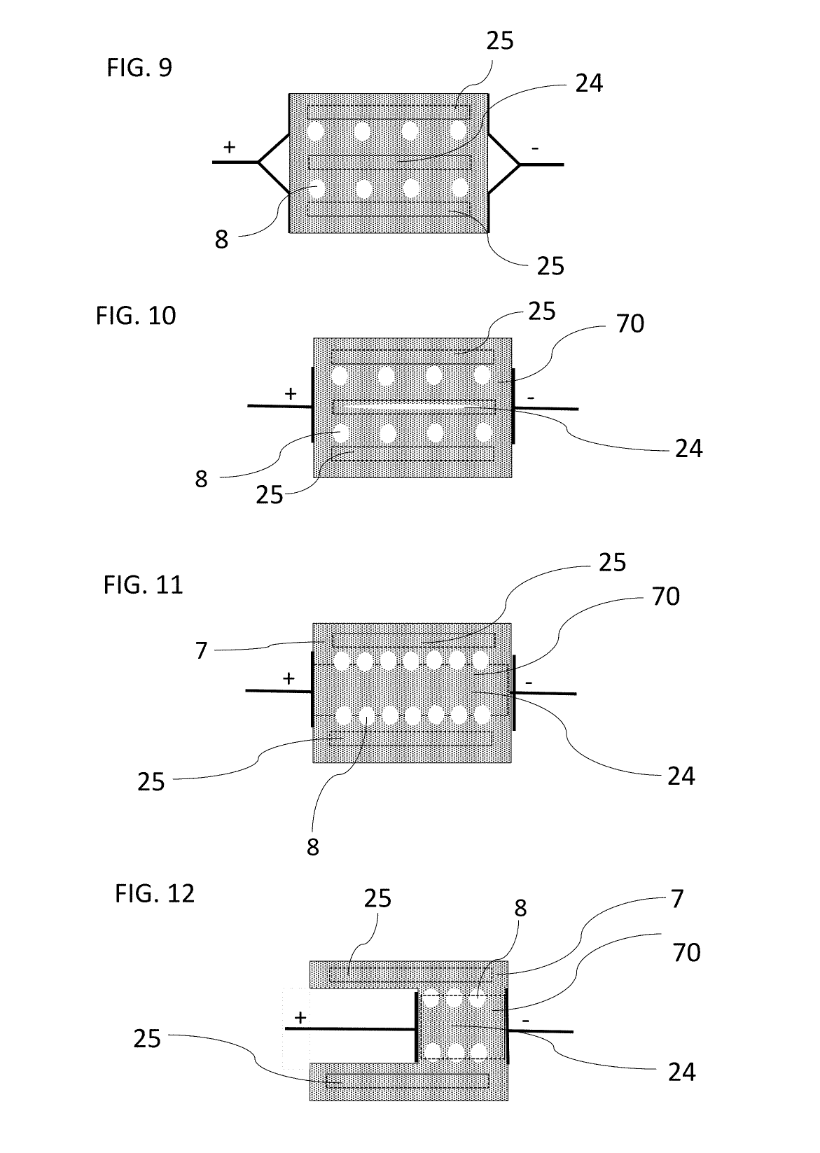

FIGS. 9 to 12 show for example cuboid or block-type vaporizers with different electronic contacting modes and illustrate control of the current flow within the vaporizer depending on the placement of the terminal contacts.

The vaporizers shown in FIGS. 9 and 10 have the same electrically conductive coating in all areas. Nevertheless, electrical currents will preferably flow only into the vaporization regions 24. The storage regions 25 remain cold, so that the liquid is not vaporized but can be stored in these regions.

In the following optimizations, electrical currents are controlled through the placement of the terminal contacts. Although all areas have the same coating, electrical currents will only flow in the vaporization region. The storage region remains cold. Liquid will not be vaporized there and can be buffered there. Hence, liquid storage and vaporization can be achieved in a single porous body. The two regions can be partially separated by vapor exit channels 8.

FIGS. 11 and 12 show embodiments in which only regions of the sintered body are provided with an electrical coating (region 70). Accordingly, only the regions 70 are electrically conductive and heatable. The outer regions 7 remain cold and serve as liquid storage and thermal insulation.

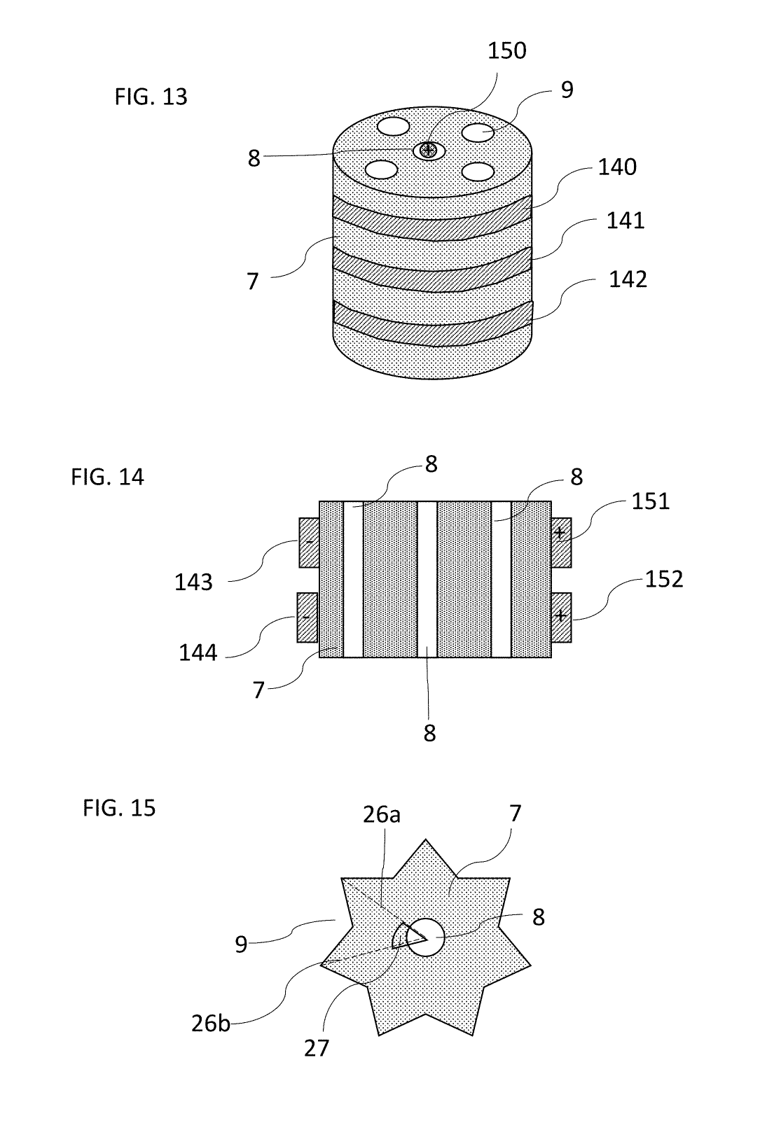

FIG. 13 shows a refinement of the invention comprising a plurality of heating zones which are defined by negative terminals 140, 141, and 142. The sintered body 7 has an electrically conductive coating, here, and has the shape of a hollow cylinder. The first channel 8 is defined by the inner circumferential surface of the hollow cylinder. It accommodates the positive terminal 150, while the second channels 9 are provided as vaporization spaces.

Negative terminals 140, 141, and 142 can be powered separately from each other. Thus, it is possible during operation to initially apply power to only one negative terminal, for example. Accordingly, the respective adjacent region of the sintered body is then heated. If, for example, a higher heating power is required during operation, the additional negative terminals can also be powered.

Alternatively, it is also possible to power all the negative terminals 140, 141, and 142, and to apply different voltages. In this manner, different heating powers can be generated at the individual negative terminals 140, 141, 142. This can be exploited to generate a gradient in the heating power within the vaporizer, for example.

FIG. 14 shows a further embodiment of the refinement according to the invention illustrated in FIG. 13. Here, the vaporizer 7 is provided in the form of a cuboid that includes channels 8. The positive terminals 151 and 152 and negative terminals 143 and 144 are located at opposite sides of the cuboid. Again, the individual terminals can be powered separately, here.

FIG. 15 shows a schematic view of a star-shaped vaporizer having a first channel 8 and a second channel 9. The second channel 9 is an open channel and is defined by the side faces of the adjacent star points. The angle x, 27, is the angle between the center lines 26a, 26b of the respective star points and correlates with the number of star points. Channel 9 may have a sharp corner so as to present a V-shaped contour, as shown in FIG. 15. However, the contour of the second channel may also be rounded.

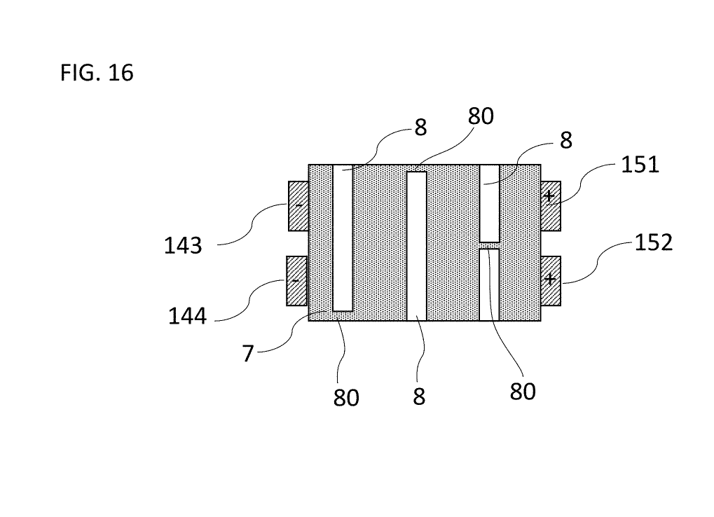

FIG. 16 shows a variant of the embodiment illustrated in FIG. 14. In the embodiment of FIG. 16, the first channels 8 are open at one end, or, more generally, the one or more channels 8 have a bottom 80 closing one end of the channel. The bottom 80 may be located at one of the end faces of the sintered body, or, as illustrated by the right-hand channel, between the open ends of the channel 8 at the end faces.

What is claimed is:

1. A liquid storage comprising a sintered body made of glass or glass ceramic, wherein the sintered body has an open porosity in a range from 10 to 90%, and wherein the sintered body has two channels, wherein the two channels are at least partially enclosed by the glass or glass ceramic of the sintered body.

2. The liquid storage of claim 1, wherein the two channels are completely enclosed by the glass or glass ceramic of the sintered body.

3. The liquid storage of claim 1, wherein the two channels comprise at least one channel having a shape selected from a group consisting of a circular bore, an ellipsoidal bore, a polygonal hole, and a slit.

4. The liquid storage of claim 1, wherein the sintered body has a length, and wherein the two channels extend over an entirety of the length of the sintered body.

5. The liquid storage of claim 1, wherein the sintered body is a cylinder having a length, and wherein a first channel of the two channels extends parallel to the length.

6. The liquid storage of claim 5, wherein the first channel is completely enclosed by the glass or glass ceramic of the sintered body and is located in a center of the cylinder.

7. The liquid storage of claim 5, wherein a second channel of two channels is completely enclosed by the glass or glass ceramic of the sintered body has a cross sectional shape selected from a group consisting of a circular, ellipsoidal, and polygonal.

8. The liquid storage of claim 5, wherein a second channel of the two channels is only partially enclosed by the glass or glass ceramic of the sintered body so that the second channel has an open longitudinal side along the length.

9. The liquid storage of claim 8, wherein the second channel has a V-shaped cross section having an angle between centerlines of the V in a range of less than or equal to 180°.

10. The liquid storage of claim 1, wherein the sintered body is a cuboid, wherein the two channels extend parallel to an edge of the cuboid.

11. The liquid storage of claim 1, wherein the two channels comprise a first channel and at least two second channels, and wherein the at least two second channels are arranged symmetrically with respect to a location of the first channel.

12. The liquid storage of claim 1, wherein the sintered body has pores with a size in a range from 1 to 5000 μm.

13. A vaporizer configured for use in an electronic cigarette, a medication administration device, and thermally heated vaporizers, comprising:
a heating element; and
a sintered body as a liquid storage, the sintered body being made of glass or glass ceramic, wherein the sintered body has an open porosity in a range from 10 to 90%, and wherein the sintered body has two channels, wherein the two channels are at least partially enclosed by the glass or glass ceramic of the sintered body.

14. The vaporizer of claim 13, wherein the heating element is disposed directly on a surface of the sintered body.

15. The vaporizer of claim 14, wherein the heating element is selected from the group consisting of a metal foil, a metal wire, and an electrically conductive coating.

16. The vaporizer of claim 15, wherein the sintered body has a length, and wherein the two channels extend parallel to the length.

17. The vaporizer of claim 16, wherein a first channel of the two channels is completely enclosed by the glass or glass ceramic of the sintered body, wherein the sintered body is a cylinder with the first channel is located in a center of the cylinder.

18. The vaporizer of claim 17, wherein a second channel of the two channels comprises at least two second channels that are arranged symmetrically with respect to the first channel.

19. The vaporizer of claim 18, wherein the electrically conductive coating is bonded to the surface area of the sintered body defined by the open pores so that when the sintered body is electrically contacted and powered, a current flows at least partially through an interior of the sintered body thereby heating the interior.

20. The vaporizer of claim 19, wherein the first channel has a surface that forms a first electrical terminal of the heating element and an outer circumferential surface of the cylinder forms a second electrical terminal of the heating element.

21. The vaporizer of claim 20, wherein the first electrical terminal is a positive terminal and the second electrical terminal is a negative terminal.

22. The vaporizer of claim 21, further comprising a third channel completely enclosed by the glass or glass ceramic of the sintered body, the third channel extending perpendicular across the cylinder, wherein the second and third channels serve as an inflow opening for fluid into the liquid storage.

23. The vaporizer of claim 22, wherein the third channel has a smaller diameter than the first channel.

24. The vaporizer of claim 21, further comprising an electrical connection of a second electrically conductive coating.

25. The vaporizer of claim 15, wherein the sintered body has a cuboid shape, wherein the two channels extend parallel to one edge of the cuboid, and wherein the two channels are completely enclosed by the glass or glass ceramic of the sintered body.

26. The vaporizer of claim 25, wherein the cuboid has two opposite side faces that extend parallel to the two channels and serve as electrical terminals.

27. The vaporizer of claim 13, wherein the heating element comprises at least two individual heating zones configured so that current flow in the two individual heating zones can be switched independently of each other.
